(19) United States
(12) Patent Application Publication
(10) Pub. No.: US 2022/0288013 A1
(43) Pub. Date: Sep. 15, 2022

(54) 1,8-CINEOL COATED IMPLANTS

(71) Applicant: THE BOARD OF TRUSTEES OF THE LELAND STANFORD JUNIOR UNIVERSITY, Stanford, CA (US)

(72) Inventors: Britta A. KUEHLMANN, Stanford, CA (US); Geoffrey C. GURTNER, Portola Valley, CA (US); Clark Andrew BONHAM, Stanford, CA (US)

(73) Assignee: THE BOARD OF TRUSTEES OF THE LELAND STANFORD JUNIOR UNIVERSITY, Stanford, CA (US)

(21) Appl. No.: 17/630,205

(22) PCT Filed: Jul. 16, 2020
(86) PCT No.: PCT/US20/42248

§ 371 Date: Jan. 26, 2022

Related U.S. Application Data
(60) Provisional application No. 62/879,379, filed on Jul. 26, 2019.

(51) Int. Cl. A61K 31/352; A61K 47/34; A61K 9/00; A61P 19/04; A61L 27/54; A61L 27/36

(52) U.S. Cl. CPC A61K 31/352; A61K 47/34; A61K 9/0024; A61P 19/04; A61L 27/54; A61L 27/3637

(57) ABSTRACT

Apparatuses, devices, compositions, and methods to ameliorate undesired side effects or outcomes, such as fibrosis due to surgical implants, wounds, or other bodily injury. In particular, described are apparatuses, devices, and compositions containing inhibitors of Wnt11 as well as methods of making and using them. These apparatuses, devices, compositions, and methods may be especially useful for preventing, reducing or treating unwanted fibrosis, such as that resulting from a biomedical implant implanted in an individual.

### CROSS REFERENCE TO RELATED APPLICATIONS

This application claims the benefit of U.S. Provisional Patent Application No. 62/879,379, filed Jul. 26, 2019, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure is related generally to ameliorating undesired side effects or outcomes such as fibrosis due to surgical implants, wounds, or other bodily injury. In particular, described herein are apparatuses, devices, and compositions containing inhibitors of Wnt11 as well as methods of making and using them. These apparatuses, devices, compositions, and methods may be especially useful for preventing, reducing or treating unwanted fibrosis, such as can result due to a biomedical implant implanted in an individual.

### BACKGROUND

Fibrosis is a common and troublesome complication following surgical implantation of biomedical device implants (such as heart valves or breast implants) into the body of an individual. Fibrosis represents a significant medical and financial burden. Fibrosis involves formation of excess connective tissue (connective tissue overgrowth), connective tissue hardening and/or scarring of connective tissue in a body. Fibrosis takes on various forms and can be extensive. An extensive foreign body response leading to fibrosis around a biomedical device that mediates remodeling of the extracellular matrix (ECM) can result in formation of a capsule of fibrotic or scar tissue that encapsulates or surrounds the implant (referred to as capsular fibrosis). Fibrosis can cause pain or discomfort, cause a lump or bump to form, and can interfere with normal tissue function. Fibrosis can even lead to implant damage to a point where the damaged implant does not work as intended, and may even need to be replaced (revision surgery). Fibrosis resulting from implantation is mediated by the body's foreign body response and can include inflammation and wound healing responses.

Existing therapies for preventing or treating fibrosis to address these and other problems are inadequate. With surgical implantation being a common procedure across many fields of medicine, having new therapeutics to optimize patient outcomes and reduce or eliminate pain, discomfort, tissue dysfunction, and damage to an implant after implantation of a biomedical device is desirable. Existing strategies for preventing or treating fibrosis are largely unsuccessful due to our insufficient understanding of the foreign body response and the pathophysiology underlying fibrosis. Recent reports from our and other laboratories have suggested that Wnt signaling plays a role in the progression of at least some of the fibrotic responses in the body and that several Wnt proteins moderate lung, liver, and skin fibrosis, among others. Activation of the Wnt pathway has been shown to increase excessive collagen fiber deposition. Some Wnt pathways have been posited to drive macrophage cells to differentiate into a pro-fibrotic profile. In this context, Wnt proteins serve as attractive and potentially promising targets for anti-fibrosis therapies. However, the Wnt family contains 18 different cell-signaling protein members made by cells in the body. Wnt family members are involved in a vast variety of different important and complicated cell processes from cell fate determination, embryonic development and inflammation to wound healing. Defective Wnt signaling is a causative factor for various pathologies, such as breast, colon, and skin cancer, and birth defects. The effects that specific Wnt protein members have on cells from one Wnt family member to the next varies.

Hence there is a critical need for therapies to improve outcomes after biomedical device implantation. Described herein are therapies for improving outcomes after biomedical device implantation using inhibitors of the Wnt11 protein.

### SUMMARY OF THE DISCLOSURE

The present invention relates to ameliorating undesired side effects or outcomes such as fibrosis due to surgical implants, wounds, or other bodily injury. In particular, described herein are apparatuses, devices, and compositions containing inhibitors of Wnt11 as well as methods of making and using them. These apparatuses, devices, compositions, and methods may be especially useful for preventing, reducing or treating unwanted fibrosis, such as can result due to a biomedical implant implanted in an individual. One aspect of the invention provides a biomedical device including an effective amount of a composition containing a Wnt11 inhibitor for preventing, inhibiting or treating capsular fibrosis. Another aspect of the invention provides a biomedical device including an effective amount of a composition containing a monoterpenoid for preventing, inhibiting or treating capsular fibrosis. Yet another aspect of the invention provides biomedical device including an effective amount of a composition containing 1,8-cineol for preventing, inhibiting or treating capsular fibrosis.

Any of these biomedical devices may include wherein the biomedical device is an implant configured to prevent, inhibit, or treat capsular fibrosis when the biomedical device is implanted into a body of an individual having or at risk of having capsular fibrosis.

In this or other biomedical devices the implant may be configured to stay in the body at least 2 weeks, at least 4 weeks, at least 8 weeks, at least 3 months, at least 6 months, or at least one year.

These or other biomedical devices may include an insert configured to prevent, inhibit, or treat capsular fibrosis when the biomedical device is inserted into a body of an individual having or at risk of having capsular fibrosis. In any of these or other biomedical devices, the biomedical implant may be an interface configured to prevent, inhibit, or treat capsular fibrosis when placed on a body of an individual having or at risk of having capsular fibrosis.

In any of these or other biomedical devices, the biomedical implant may be configured to administer the composition systemically. In any of these or other biomedical devices, the biomedical implant may be configured to administer the composition locally.

In any of these or other biomedical devices, the biomedical implant may be configured to administer the composition intraarterially, intraarticularly, intramuscularly, intraocularally, intraperitoneally, intravenously, or subcutaneously. In these or other biomedical devices, the biomedical implant may be configured to administer the composition buccally, intranasally, orally, intrarectally, intravaginally, or sublingually. In these or other biomedical devices, the biomedical implant may be configured to administer the composition iontophoretically, topically, or transdermally.

In these or other biomedical devices, the biomedical implant may be coated with the composition. In these or other biomedical devices, the biomedical implant may be impregnated with the composition. In these or other biomedical devices, the composition may be covalently attached to the device. In these or other biomedical devices, the composition may be non-covalently attached to the device. In these or other biomedical devices, the biomedical device may include a silicone implant.

Yet another aspect of the invention provides a method of preventing, inhibiting, or treating capsular fibrosis in an individual in need thereof including the steps of selecting an individual that has or is at risk of having fibrosis; and administering to the individual a composition having an effective amount of a Wnt11 inhibitor and thereby preventing, inhibiting, or treating capsular fibrosis in the individual.

Yet another aspect of the invention provides a method of preventing, inhibiting, or treating capsular fibrosis in an individual in need thereof including the steps of selecting an individual that has or is at risk of having capsular fibrosis; and administering to the individual a composition having an effective amount of a monoterpenoid and thereby preventing, inhibiting, or treating capsular fibrosis in the individual.

Yet another aspect of the invention provides a method of preventing, inhibiting, or treating capsular fibrosis in an individual in need thereof including the steps of selecting an individual that has or is at risk of having capsular fibrosis; and administering to the individual a composition having an effective amount of 1,8-cineol and thereby preventing, inhibiting, or treating capsular fibrosis in the individual. Yet another aspect of the invention provides a method of preventing, inhibiting, or treating capsular fibrosis in an individual in need thereof including the steps of selecting an individual that has or is at risk of having capsular fibrosis; and administering to the individual a composition having an effective amount of macrophage inhibiting activity and thereby preventing, inhibiting, or treating capsular fibrosis in the individual.

Yet another aspect of the invention provides a method of preventing, inhibiting, or treating capsular fibrosis in an individual in need thereof including the steps of selecting an individual that has or is at risk of having capsular fibrosis; and administering to the individual a composition having an effective amount of an M2 macrophage inhibiting activity and thereby preventing, inhibiting, or treating capsular fibrosis in the individual.

In these or any other methods the composition may be administered intraarterially, intraarticularly, intramuscularly, intraocularally, intraperitoneally, intravenously, or subcutaneously. In these or any other methods the composition may be administered buccally, intranasally, orally, intrarectally, intravaginally, or sublingually. In these or any other methods the composition may be administered iontophoretically, topically, or transdermally.

In these or any other methods the composition may be administered locally. In these or any other methods the composition may be administered systemically.

In these or any other methods the administering step includes implanting an implant including the composition.

In these or any other methods the implant is further configured to treat a medical issue or cosmetic issue other than fibrosis.

In these or any other methods the administering step includes inserting an insert including the composition. In these or any other methods the administering step includes placing a skin interface of a biomedical device on a skin of the individual.

In these or any other methods the composition prevents, inhibits, or reduces macrophage activity or differentiation by at least 10%, at least 20%, at least 30%, at least 40% or at least 50%. In these or any other methods the composition prevents, inhibits, or reduces M2 macrophage activity or differentiation at least 10%, at least 20%, at least 30%, at least 40% or at least 50%. In these or any other methods the composition prevents, inhibits, or reduces Cd36 macrophage activity or differentiation, Ccr2 macrophage activity or differentiation and/or Cd209 macrophage activity or differentiation at least 10%, at least 20%, at least 30%, at least 40% or at least 50%.

In these or any other methods the composition prevents, inhibits, or treats collagen deposition. In these or any other methods the composition prevents, inhibits, or treats a foreign body response.

In these or any other methods the composition the individual is human.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A, 1B, 1C and 1D show hematoxylin and eosin (H&E) staining of sections from Bl6 vs Wnt11 knockout mice, 3 months post-implantation of an implant. The capsule formed around the Wnt11 knockout-mice is significantly thinner with a lower cellularity and more loosely arranged fibers compared to the capsule of Bl6-mice.

FIGS. 2A, 2B, 2C, 2D, 2E, 2F, 2G, 2H, and 2I show unique immune cell subsets during the progression of capsular fibrosis. FACS analysis of the murine capsule displayed significant reductions in the macrophage populations (Cd36, Cd209+, Ccr2+) of the capsules of Wnt11 knockout mice compared to Bl6 control mice.

### DETAILED DESCRIPTION

### Definitions

Fibrosis. Fibrosis is the formation of excess fibrous connective tissue, such as overgrowth, hardening and/or scarring of connective tissue. It is thought to be due to an excess deposition of extracellular matrix components such as collagen and glycosaminoglycans, usually as a result of injury. Capsular fibrosis is fibrosis that forms a capsule around or encloses an object such as an implant.

Wnt11. Wnt11 is a protein member of the Wnt family. (See Lako M, Strachan T, Bullen P, Wilson D I, Robson S C, Lindsay S (December 1998). “Isolation, characterization and embryonic expression of WNT11, a gene which maps to 11q13.5 and has possible roles in the development of skeleton, kidney and lung”. Gene. 219 (1-2): 101-10. doi: 10.1016/S0378-1119(98)00393-X). The Wnt family includes at least 18 secreted cell-signaling proteins. Wnt proteins direct signal transduction through canonical (ß-catenin dependent) or non-canonical pathways. In a canonical (ß-catenin dependent) Wnt signaling pathway, Wnt signaling inhibits the degradation of ß-catenin which regulates transcription of a number of genes in the cytosol of the cell. In the canonical (ß-catenin dependent) pathway, Wnt signaling is activated via binding of a Wnt protein to its respective dimeric cell surface receptor (seven transmembrane frizzled Wnt receptor proteins (Fzd) and LRP5/6 co-receptor proteins). Activation of frizzled leads to activation of the protein Dishevelled (Dv1) in the cytosol which regulates transcription of a number of genes. In one proposed non-canonical Wnt signaling pathway, Wnt signaling is activated via binding of a Wnt protein to its respective dimeric cell surface receptor (seven transmembrane frizzled Wnt receptor proteins (Fzd)). In another proposed non-canonical Wnt pathway, Wnt signaling is activated via binding of a Wnt protein to its respective dimeric cell surface receptor (seven transmembrane frizzled Wnt receptor proteins (Fzd)) and Wnt protein modulates the levels of calcium inside the cell. Without wishing to be limited to a particular mechanism, inhibition of Wnt11 may lead to inhibition via a non-canonical or, more commonly, a canonical Wnt pathway.

Monoterpenoid. A monoterpenoid is a small molecule having a 10 carbon backbone structure with 2 linked isoprene units (with a branched-chain unsaturated hydrocarbon; 2-methyl-1,3-butadiene). Monoterpenoids can generally be divided into three subgroups: acyclic, monocyclic, and bicyclic and may be modified by oxidation, methylation, or glycosylation. Examples of aliphatic monoterpenoids include myrcene, citral, geraniol, lavandulol, and linalool. Examples of monocyclic monoterpenoids include α-terpineol, limonene, thymol, menthol, carvone, eucalyptol, and perillaldehyde. Examples of bicyclic monoterpenes include thujone, Δ3-carene, α-pinene, β-pinene, borneol and camphor.

1,8-cineol. 1,8-cineol, also known as cineol, cineole, 1,8-cineol, 1,8-cineole, eucalyptol, and 1,3,3-Trimethyl-2-oxabicyclo[2.2.2]octane, is a naturally occurring monoterpenoid (cyclic ether) found in various species of plants, including in Eucalyptus trees (e.g., Eucalyptus globulus leaves) and Salvia plants. A cyclic ether and colorless liquid, it has a formula of C₁₀H₁₈0 and is generally referred to an “essential oil”. 1,8-cineol may be obtained from extracts from plants or parts of plants such as leaves that contain it (a “native extract”) or may be produced through other means such as naturally occurring or modified bacteria or fungi or chemical synthesis. 1,8-cineol is often the main component of plant-based extracts and may be used as extracted or may be further purified prior to use. 1,8-cineol has been used as an anti-cancer, anti-bacterial, and anti-inflammatory agent. 1,8-cineol leads to decreased activity of the pro-inflammatory NF-κB-signaling. Additionally, exposure of different cell lines to 1,8-cineol treatment has been shown to inhibit proliferation and to decrease the activity of the WNT/β-catenin pathway (e.g., the canonical pathway), especially of Wnt11, in permanent head and neck squamous cell carcinoma (HNSCC) cell lines. It also reduces expression of Wnt11 in these cell lines (Roettger A et al., Inhibitory Effect of 1,8-Cineol on β-Catenin Regulation, WNT11 Expression, and Cellular Progression in HNSCC. Front Oncol. 2017; 7: 92. doi: 10.3389/fonc.2017.00092). To our knowledge, 1,8-cineol has been described to act as an inhibitor of the Wnt/β-catenin activity in only HNSCC (Head and neck squamous cell carcinoma) via a decreased inhibition of GSK-3 (glycogen synthase kinase 3). This leads to reduced levels of WNT11 (Roettger A et al.). Additionally, 1,8-cineol has been mentioned to prophylactically and therapeutically treat nose polyps (Polyposis nasi et sinuum).

Macrophage inhibiting activity. Macrophages are a heterogeneous population of cells in the body. Derived from monocytes, a type of white blood cell, they undergo differentiation in response to specific signals and act to maintain and restore homeostasis in an individual. Macrophage inhibiting activity prevents or reduces the differentiation or activity of macrophages.

M2 macrophage inhibiting activity. As indicated above, macrophages are derived from monocytes. Macrophages can be divided into M1 and M2 macrophage subsets based on their specific activation and the specific differentiation they undergo. Activated M1 macrophages (sometimes referred to as “classically” activated macrophages), are characterized by the production of high levels of pro-inflammatory cytokines and an ability to mediate resistance to pathogens. M2 macrophages (sometimes referred to as “alternatively” activated macrophages) are associated with wound healing and tissue repair, and contribute to the formation of extracellular matrix (e.g., fibrous tissue). M2 macrophages are activated by exposure to certain cytokines, such as IL-4, IL-10, or IL-13.

Devices and Methods

Described herein are apparatuses (systems and devices including compositions) and methods of making and using them for preventing, inhibiting, or treating fibrosis and preventing, inhibiting, or treating excess or unwanted fibrous tissue in an individual. In particular described herein are apparatuses (systems and devices) and compositions containing inhibitors of Wnt11 protein, as well as methods of making and using them. These apparatuses and compositions may be particularly useful for preventing, inhibiting, or treating (reducing) excess fibrous connective tissue such as that caused by injury or implantation of a medical device into a tissue of an individual.

One aspect of the invention describes a biomedical device including an effective amount of a composition containing a Wnt11 inhibitor for preventing, inhibiting or treating fibrosis, and in particular for preventing, inhibiting or treating capsular fibrosis. A Wnt11 inhibitor as described for use herein may be a monoterpenoid, such as an acyclic monoterpenoid, a monocyclic monoterpenoid, and a bicyclic monoterpenoid, modified by oxidation, methylation, or glycosylation and combinations thereof. Examples of aliphatic monoterpenoids considered include myrcene, citral, geraniol, lavandulol, and linalool. Examples of monocyclic monoterpenoids considered include α-terpineol, limonene, thymol, menthol, carvone, eucalyptol, and perillaldehyde. Examples of bicyclic monoterpenes considered include thujone, Δ3-carene, α-pinene, β-pinene, borneol and camphor. In a particular example, a Wnt11 inhibitor as described for use herein for preventing, inhibiting or treating fibrosis is 1,8-cineol. In some embodiments, a Wnt11 inhibitor such as a monoterpenoid (such as 1,8-cineol) as described herein inhibits a canonical Wnt pathway in a target cell. In some embodiments, a Wnt11 inhibitor such as a monoterpenoid (such as 1,8-cineol) as described herein inhibits a non-canonical Wnt pathway in a target cell (e.g., a planar cell polarity (PCP) pathway or a calcium pathway).

Fibrosis may be prevented, inhibited, or treated with a Wnt11 inhibitor such as a monoterpenoid (e.g., 1,8-cineol) implanted in, inserted in, or placed on the body of an individual in need thereof, such as an individual having or at risk of having fibrosis. A biomedical device as described herein for preventing, inhibiting, or treating fibrosis may be configured for implanting into a body of an individual, for inserting into a body of an individual, or for placing on a body of an individual, the individual having or at risk of having fibrosis. A biomedical device may be configured to replace or repair damaged tissue structures or to prevent or reduce the risk of damaging tissue structures.

In some embodiments, a biomedical device as described herein for administering a Wnt11 inhibitor such as a monoterpenoid (e.g., 1,8-cineol) may be an implantable biomedical device (e.g., may be an implant) and may be configured (sized and shaped) for implanting into an individual, such as being configured for implanting intraarterially, intraarticularly, intramuscularly, intraocularally, intraperitoneally, intravenously, or subcutaneously. A biomedical device as described herein for administering a Wnt11 inhibitor such as a monoterpenoid (e.g., 1,8-cineol) may be an implantable biomedical device (e.g., may be an implant) configured to administer the Wnt11 inhibitor such as a monoterpenoid (e.g., 1,8-cineol) intraarterially, intraarticularly, intramuscularly, intraocularally, intraperitoneally, intravenously, or subcutaneously. In some embodiments, the biomedical device is a breast implant, a catheter, a coil, a heart valve, a lens, a prosthesis, or a stent. A biomedical device may serve as a depot for one or more drugs, which may include a monoterpenoid (e.g., 1,8-cineol) and may include one or more other drugs.

In some embodiments, a biomedical device as described herein for administering a Wnt11 inhibitor such as a monoterpenoid (e.g., 1,8-cineol) may be an insertable biomedical device (e.g., be an insert) and may be configured (sized and shaped) for inserting into an individual, such as into or through a naturally occurring orifice, such as into or through an anus/colon/rectum, an ear (ear canal), an esophagus, a mouth, a nose (a nares), or a vagina. In general such an insertable biomedical device is expellable or readily removable from the body of the individual (and especially without requiring surgery to do so) and may be configured to be expellable or removable from the body of the individual after 1 day, after 2 days, after 3 days, or after a week. In some embodiments, an insertable (or any other) biomedical device as described herein may be biodegradable. A biomedical device as described herein may be an insertable biomedical device and may be configured to administer a composition such as a Wnt11 inhibitor such as a monoterpenoid (e.g., 1,8-cineol) buccally, intranasally, orally, intrarectally, intravaginally, or sublingually.

In other embodiments, a device may be configured (sized and shaped) for placing on a body of an individual, such as on the skin of the individual. In some embodiments, a device may be a gas, a gel, a liquid, or a solid. A device configured to administer a composition such as a Wnt11 inhibitor such as a monoterpenoid (e.g., 1,8-cineol) for placing on a body of an individual may be a composition, a dressing (including a wound dressing), or a patch. A device for placing on a body of an individual may be configured for topical delivery or transdermal delivery. In some embodiments, a device configured to administer a composition as described herein may be configured for assisted delivery of the inhibitor, such as by iontophoresis or microinjection (such as with one or a plurality of microneedles). A biomedical device as described herein may be configured for administering a composition (a Wnt11 inhibitor such as a monoterpenoid (e.g., 1,8-cineol) ionotophoretically, topically, or transdermally.

In some embodiments, a medical device having a Wnt11 inhibitor is configured to stay in the body at least 2 weeks, at least 4 weeks, at least 8 weeks, at least 3 months, at least 6 months, or at least one year.

In any of the embodiments described herein, the Wnt11 inhibitor composition may be coated on an outer surface of a biomedical device or the Wnt11 inhibitor composition may be impregnated in the biomedical device, or may be both coated on an outer surface and impregnated in the device. A coating may be, for example, from 0.1 um thick (or less) to 10 um thick. In any of the embodiments described herein, the Wnt11 inhibitor composition may be covalently attached (e.g., through a covalent bond) to the medical device or may be non-covalently attached (e.g., through non-covalent interactions such as van der Waals forces or hydrogen bonding) or may be both covalently attached and non-covalently to the medical device. A Wnt11 inhibitor may act on the body of an individual while attached to a device or may act after detaching (being detached from a device). A biomedical device as described herein for administering a Wnt11 inhibitor may be configured to release inhibitor from the biomedical device (such as into a bloodstream or colon) or may be configured to hold inhibitor and not release it or may be configured to do both. Thus a Wnt11 inhibitor may be delivered locally (e.g., effectively delivered at or close to the Wnt11 delivery site) and/or may be delivered beyond the local site, such as delivered systemically. For example, a biomedical device with covalently attached Wnt11 inhibitor may be configured to not release the Wnt11 inhibitor and the composition on the device may be configured to act locally. In other examples, a biomedical device with non-covalently attached Wnt11 inhibitor may release or be configured to release its payload of Wnt11 inhibitor and an effective amount of a Wnt11 inhibitor may only travel a short distance, such as less than 2, less than 5, less than 10 or less than 20 times the longest dimension of the implant (e.g., if the biomedical device is an implant that is 5 mm long in the longest dimension, the device and Wnt11 attached thereto may be configured such that an effective amount Wnt11 inhibitor only travels 10 mm (e.g., 2 times the longest dimension of the implant) away from the implant. In other examples, a biomedical device with non-covalently attached Wnt11 inhibitor may be configured to release its payload of Wnt11 inhibitor and released Wnt11 inhibitor may travel a significant distance through the body of an individual, such as through a bloodstream or skin. In some embodiments, a biomedical device may be configured to administer a Wnt11 inhibitor locally. In some particular embodiments, a biomedical device may be an implant for implanting into an individual in need thereof and may be configured to administer a Wnt11 inhibitor locally. In some embodiments, a biomedical device may be configured to release some or all of its payload of Wnt11 inhibitor from the device. In some embodiments a device having a Wnt11 inhibitor may be configured for releasing (administering) some or all of its Wnt11 inhibitor payload systemically. For example, a device having a Wnt11 inhibitor may be configured for placing in a blood vessel and releasing or administering its Wnt11 inhibitor payload systemically. A Wnt11 (e.g., 1,8-cineol) coating on a device may be a uniform coating or a non-uniform coating. In some embodiments, a coating may be applied to the surface of a biomedical device without the use of an inert polymer. For example, exemplary embodiments of the drug releasing coatings described herein do not require that the application of an inert polymer layer to the surface of the medical device to bind a therapeutic agent (e.g., 1,8-cineol) to the implant's surface. A Wnt11 inhibitor (e.g., 1,8-cineol) coating) on the surface of a medical device or impregnated into a device may be attached using a binder. A binder is used to impart cohesive qualities to a composition, and thus ensure that a composition remains intact (as desired) after cohesion of the composition to a biomedical device. In any of the embodiments described herein, a coating composition may be composed of at least one therapeutic agent (e.g., 1,8-cineol) in a composition dispersed in a modified, biologically active binders. The therapeutic agent in a coating composition or an impregnation composition may be 1,8-cineol and may further include another therapeutic agent, such as anti-inflammatory drug, such as dexamethasone to inhibit inflammation. In some embodiments, no additional therapeutic agent is included in a coating composition or impregnation composition. In some particular examples, a therapeutic agent will be applied to the surface of a biomedical device (e.g., an implantable device) via covalent bonding. In some embodiments, a coating of monoterpenoid (e.g., 1,8-cineol) may be a “cap coating”. A “cap coating” may be applied over a therapeutic agent or drug releasing coating. A “cap coating” may act or may be configured to act as a barrier. A “cap coating” may control or may be configured to control release of the therapeutic agent or drug releasing coating (and for bioactive binders) from a surface of a biomedical device. A cap coating may include a Wnt11 inhibitor such as a monoterpenoid (e.g., 1,8-cineol) and may include polymers (e.g., silicone-based polymers) having Wnt11 inhibitor such as a monoterpenoid (e.g., 1,8-cineol). A cap coating may have elastomeric properties. In some embodiments, a cap coating, such as one with elastomeric properties, may allow or may be configured to allow the cap coating to be applied to an expandable or flexible medical devices. Accordingly, the elastic properties of the cap coating may permit the coating to be expanded and flexed without comprising the integrity of the cap coating and thereby allowing for the controlled release of therapeutic agents (and biologically active binders) from the surface of the implant. Accordingly, the controllable release of these components at the site of implantation may treat, reduce or prevent pathologies associated with the implantation of the device. Additionally, the therapeutic agents (and biologically active binders) may be controllably released from the surface of the medical implant by providing a cap coating on the biomedical device, and especially on a biomedical implant device. A biomedical device may have a Wnt11 inhibitor, such as a monoterpenoid (e.g., 1,8-cineol) covalently bonded thereto. Wnt11 inhibitor, such as a monoterpenoid (e.g., 1,8-cineol) may be covalently bonded by physically blending or dispersing it with a polymers such as inert polymer. Such an inert polymers may not possess any known pharmacological activity and in some cases may only serve as a carrier or binder for Wnt11 inhibitor, such as a monoterpenoid (e.g., 1,8-cineol). The use of inert polymers for coating may allow larger doses of drugs to be applied to the medical device surface and concomitantly larger amounts of the drug may be released and especially with less toxicity.

A biomedical device as described herein is generally biocompatible and is made from a biocompatible material. A biomedical device as described herein may be either biodegradable or may be non-biodegradable or may be a combination in which a first part is biodegradable and a second part is non-biodegradable. The biomedical device may be made from, including being coated or layered with, acrylic, hydroxyethyl methacrylate (HEMA), methacrylate, polyamine, polycaprolactone, polyglycolic acid, polyester, polyether, polypropylene, polysiloxane, polyurethane, polylactic acid, silicone, another biodegradable or non-biodegradable co-polymer or polymer, a metallic material, a non-metallic material, or combinations thereof. In a particular example, the biomedical device is made from a biocompatible silicone. In some examples, a biomedical device may contain many particles, such as a plurality of nanoparticles containing Wnt11 inhibitor.

Another aspect of the invention provides a method of preventing, inhibiting, or treating fibrosis (e.g., capsular fibrosis) in an individual in need thereof. Any of these methods may include selecting an individual that has or is at risk of having fibrosis (e.g., capsular fibrosis). An individual that has fibrosis (e.g., capsular fibrosis) may, for example, have fibrosis encapsulating a previously placed biomedical device (implant) as a result of receiving the device implant). An individual at risk of having fibrosis may be an individual who is having or planning to have a biomedical device (implant) placed in the body of the individual and there may be a risk that fibrosis and especially capsular fibrosis occurs around the implant. Any of these methods may include administering to the individual a composition having an effective amount of a Wnt11 inhibitor and may include thereby preventing, inhibiting, or treating capsular fibrosis in the individual. Any of these methods may include administering to the individual a composition having an effective amount of 1,8-cineol, and may include thereby preventing, inhibiting, or treating capsular fibrosis in the individual. Any of these methods may include administering to the individual a composition having an effective amount of macrophage inhibiting activity and may include thereby preventing, inhibiting, or treating capsular fibrosis in the individual. Any of these methods may include administering to the individual a composition having an effective amount of an M2 macrophage inhibiting activity and may include thereby preventing, inhibiting, or treating capsular fibrosis in the individual. In some embodiments, the amount of Wnt11 inhibitor administered may be titrated to effectively allow no or little fibrosis. In some embodiments, the amount of Wnt11 inhibitor administered may be titrated to effectively allow some fibrosis. For example, fibrosis can be a means for anchoring and holding a biomedical device (implant) so that it remains in or close to an implant location.

Any of these methods may include wherein the composition (Wnt11 inhibitor) is administered intraarterially, intraarticularly, intramuscularly, intraocularally, intraperitoneally, intravenously, or subcutaneously. Any of these methods may include wherein the composition is administered buccally, intranasally, orally, intrarectally, intravaginally, or sublingually. Any of these methods may include wherein the composition is administered iontophoretically, topically, or transdermally. Any of these methods may include wherein the composition is administered systemically. For example, a composition may be non-covalently attached to a biomedical device and released from the biomedical device, such as into skin or a bloodstream of the individual. Any of these methods may include wherein the composition is administered locally. For example, a composition may be covalently attached to a biomedical device (an implant). In any of these or other biomedical devices the composition may remain attached or may be released locally, such that it is configured and able to act at or near the site of implantation of the implant. Such attached or locally released composition may advantageously have a relatively high concentration of the composition for preventing, reducing, or treating fibrosis while preventing or reducing unwanted effects further away from the site of implantation. As indicated elsewhere herein, Wnt11 can have pleiotropic effects in the body and in some examples it may be desirable to maintain the composition locally.

In any of the methods described herein wherein the biomedical device comprising the composition is an implant, the method may further include the step of making an incision in the individual and implanting the implant into the individual through the incision. In any of these methods or devices described herein, the implant may be further configured to treat a medical issue or cosmetic issue other than fibrosis, such as a breast issue, a dental issue an ear or hearing issue, an eye or sight issue, a heart issue, a joint issue, a skin issue, a spinal issue, or a uterine issue. In some embodiments, an issue as addressed by the methods or devices herein may be due to reduced or loss of function (such as a damaged spinal disc). In some embodiments, an issue as addressed by the methods or devices herein may be due to excess or unwanted tissue or tissue function (such as a tumor). In some embodiments, an issue as addressed by the methods or devices herein may be an enhancement or an enlargement, such as a breast enhancement or breast enlargement. An implant configured to administer or deliver a Wnt11 inhibitor may be an artificial eye lens, a breast implant, a cardioverter defibrillator, a cochlear implant, a dental implant, an ear tube, an intra-uterine device, a metal implant, a pacemaker, a silicone implant, a spinal implant (e.g., an artificial disc, rod, screw), a surgical mesh, a shunt, or a stent. Any of these implants may be coated with and/or impregnated with a Wnt11 inhibitor including any monoterpenoid (e.g., 1,8-cineol) as indicated elsewhere herein.

In any of the methods described herein wherein the biomedical device including the composition is an insert, the method may further include inserting the insert into an orifice of the individual as indicated elsewhere herein. In any of the methods described herein wherein the biomedical device including the composition is or includes a skin interface, the method may further include placing the skin interface on a skin of the individual. In any of the methods or devices described herein the composition prevents, inhibits, or reduces macrophage activity or differentiation or is configured to prevent, inhibit, or reduce macrophage activity or differentiation by at least 10%, at least 20%, at least 30%, at least 40% or at least 50%, such as when compared with a method or device not having the Wnt11 inhibitor such as not having a monoterpenoid (e.g., 1,8-cineol). In any of the methods or devices described herein the composition prevents, inhibits, or reduces M2 macrophage activity or differentiation or is configured to prevent, inhibit, or reduce M2 macrophage activity or differentiation by at least 10%, at least 20%, at least 30%, at least 40% or at least 50% such as when compared with a method or device not having the Wnt11 inhibitor such as not having a monoterpenoid (e.g., 1,8-cineol).

In any of the methods or devices described herein the composition prevents, inhibits, or reduces Cd36 macrophage activity or differentiation, Ccr2 macrophage activity or differentiation and/or Cd209 macrophage activity or differentiation or is configured to prevent, inhibit, or reduce Cd36 macrophage activity or differentiation, Ccr2 macrophage activity or differentiation and/or Cd209 macrophage activity or differentiation, by at least 10%, at least 20%, at least 30%, at least 40% or at least 50% such as when compared with a method or device not having the Wnt11 inhibitor such as not having a monoterpenoid (e.g., 1,8-cineol).

In any of the methods or devices described herein the composition prevents, inhibits, or treats or is configured to prevent, inhibit, or treat collagen deposition e.g., by at least 10%, at least 20%, at least 30%, at least 40% or at least 50% such as when compared with a method or device not having the Wnt11 inhibitor such as not having a monoterpenoid (e.g., 1,8-cineol). In any of the methods or devices described herein the composition prevents, inhibits or treats a foreign body response or is configured to prevent, inhibit or treat or e.g., by at least 10%, at least 20%, at least 30%, at least 40% or at least 50% such as when compared with a method or device not having the Wnt11 inhibitor such as not having a monoterpenoid (e.g., 1,8-cineol).

In any of the methods or devices described herein the individual may be an animal, such as a human, a domestic or farm animal such as a cat, cow, dog or pig, or a wild animal.

Another aspect of the invention provides a composition comprising an effective amount of a Wnt11 inhibitor such as a monoterpenoid (e.g., 1,8-cineol). An effective amount of a Wnt11 inhibitor such as a monoterpenoid (e.g., 1,8-cineol) may be an amount effective to prevent, reduce or treat fibrosis (capsular fibrosis) and/or to prevent, inhibit, reduce or treat any of the conditions described herein (such as to prevent, inhibit, or reduce macrophage activity or differentiation, prevent, inhibit, or reduce macrophage M2 activity or differentiation, prevent, inhibit, or treat collagen deposition, a prevent, inhibit, or treat foreign body response) such as at least 10%, at least 20%, at least 30%, at least 40% or at least 50% or not more than 10%, 20%, 30%, 40%, or 50% than such as when compared with a vehicle not having the Wnt11 inhibitor such as not having a monoterpenoid (e.g., 1,8-cineol).

### Experimental

Targeting Wnt11 Reduces Fibrosis Around Implants

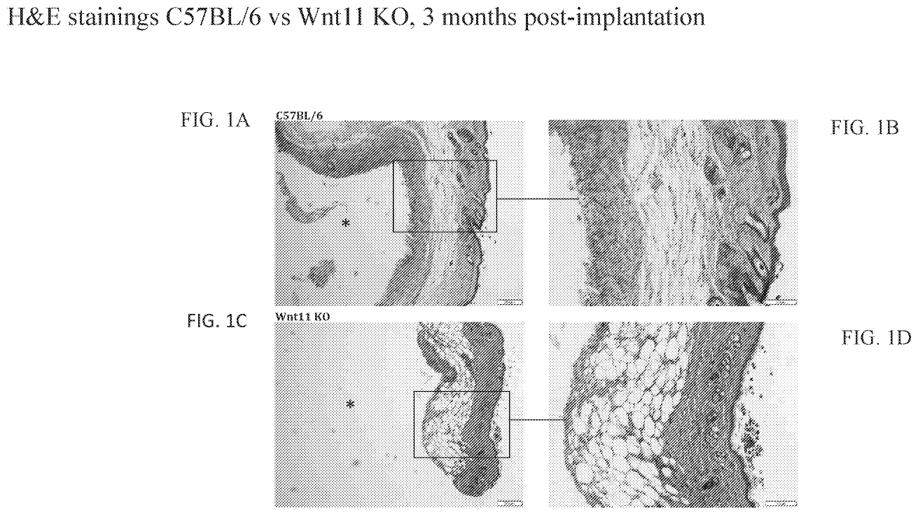

FIGS. 1A-1D shows hematoxylin and eosin (H&E) staining of sections from Bl6 mice vs Wnt11 knockout mice, 3 months post-implantation of an implant. Histological sections of the murine capsule including skin stained with H&E display the extent of capsule development. * shows the implant side. FIGS. 1A-1B shows capsule formed around implant of Bl6-mouse after 3 months, FIGS. 1C-1D shows capsule formed around implant of Wnt11 knockout-mouse after 3 months. 4× magnification (scale bar: 200 μm) FIG. 1A, FIG. 1C (left), boxes indicate the second images (FIG. 1B, FIG. 1D) with 10× magnification (scale bar: 100 μm). The capsule formed around the Wnt11 knockout mice is significantly thinner with a lower cellularity and more loosely arranged fibers compared to the capsule of C57BL/6 mice. Both capsules show morphologies consistent with synovial metaplasia. The Wnt11 knockout (KO) mice displayed a significant decrease in fibrotic deposition characterized by a significant reduction of collagen in the capsules with lower cellularity and more loosely arranged fibers than did the capsules of Bl6 control mice after 3 months. The histochemical analyses confirmed that Wnt11 knockout mice developed a significantly thinner capsule in terms of collagen fiber density, organization and cellularity underneath the subcutaneous layer than Bl6 mice.

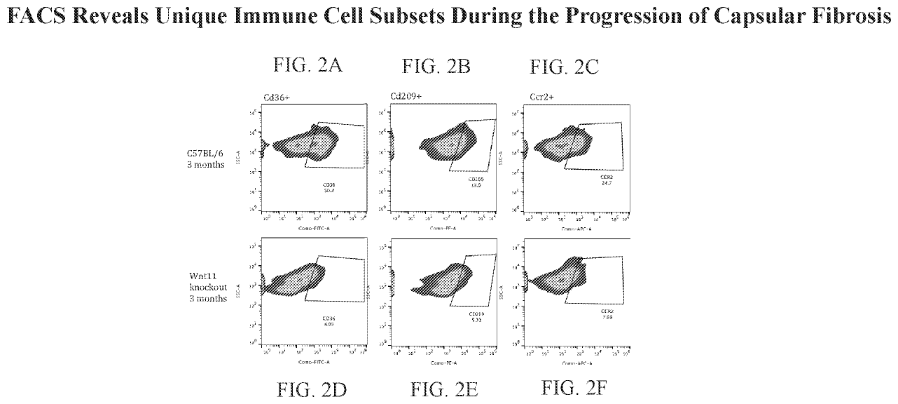

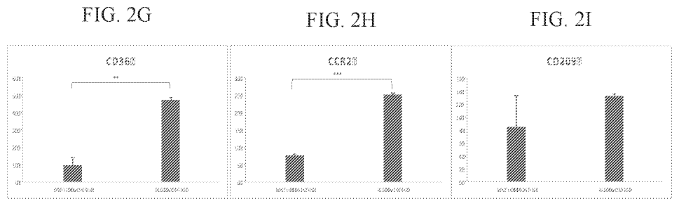

FIGS. 2A-2B. FIGS. 2A-2B show fluorescence activated cell sorting (FACS) analysis of unique immune cell subsets during the progression of capsular fibrosis. FACS analysis of the murine capsule displayed reductions in the macrophage populations (Cd36, Cd209, Ccr2+) of the capsules of Wnt11 knockout mice compared to Bl6 mice. The results display significant reductions in macrophage subpopulations, labeled by their expression of the profibrotic markers Cd36, Ccr2+ and Cd209. Wnt11 knockout mice showed a lower expression of these markers, with Cd36 and Ccr2+ both being significantly reduced. Cd209 was also decreased in the capsule of Wnt11 KO mice—statistical hypothesis testing does not conclude it was a significant decrease. This is believed mainly due to the fact that the limited data is skewed due to an outlier.

FACS and immunohistochemistry stains revealed that collagen-depositing macrophages are responsible for the development of implant fibrosis. qPCR revealed that the majority of the cells in the fibrotic tissue expressed macrophage genes and were found to significantly increase Col1a1 and Wnt11. We looked at an intervention to reduce macrophages by using Wnt11 knockout mice and subsequent induction of fibrosis. Using a murine model to simulate the generation of capsular fibrosis (CF) around a breast implant, we compared its development within Wnt11 knockout mice to that of regular C57BL/6 mice, finding a significant decrease in fibrotic deposition (capsules) in the experimental group. Fibrosis was induced by inserting implants in C57BL/6 mice and Wnt11 knockout mice. H&E staining was done to qualitatively assess the morphology and density of the fibrotic tissues. Cells were isolated and characterized by fluorescence-activated cell sorting (FACS), single cell quantitative polymerase chain reaction (qPCR), and single cell RNA sequencing to determine their impact on fibrotic responses. Additionally, scanning electron microscopy (SEM), transmission electron microscopy (TEM), and 3D confocal imaging were performed. Wnt11 knockout mice displayed a loss in certain macrophage subpopulations defined by pro-fibrotic markers (Cd36, Ccr2 and Cd209). Wnt11 knockout mice displayed significantly thinner capsules with loosely arranged fibers after 3 months compared to the capsules of regular C57BL/6 mice. These findings show that Wnt signaling and Wnt11's role specifically are important to the progression of capsular fibrosis and strongly support that Wnt11 inhibition is applicable for use in therapeutics for reducing fibrosis.

Wnt11 knockout and C57BL/6 mice (6-week old female) were used and divided into two groups with C57BL/6 mice serving as the control group. Both groups received smooth silicone implants. A 2 cm longitudinal paravertebral incision was performed under sterile conditions. At days 15, 30 and 90 after insertion of the silicone device, mice in both groups were euthanized and the implants including the surrounding capsules were harvested en-block for further analysis, such as FACS analysis and single cell RNA sequencing. Using transcriptional analysis we found distinct markers to differentiate three macrophage subgroups (Group 1=Cd36, Group 2=Cd209, Group 3=Ccr2). Capsules of Wnt11 knockout mice and C57BL/6 mice at 15 days, 30 days and 90 days post-implantation were subjected to fluorescence activated cell scanning (FACS) analysis to determine the cells responsible for the fibrotic response. These capsules were immune-stained with Cd45, Cd11b and F4/80 to determine if the immune cells were myeloid cells or lymphocytes. On all days (day 15, day 30 and day 90 after insertion), the cells were predominantly Cd45+/Cd11b+ myeloid cells and expressed the macrophage marker F4/80. We found reductions in the macrophage subgroups defined previously following FACS analysis of the capsule in Wnt11 knockout mice. The Wnt11 knockout capsules (3 months) displayed a notable decline in the macrophage subpopulations, with Cd36 and Ccr2 both being significantly reduced, while Cd209 was reduced though not significantly compared to C57BL/6 mice. Our findings suggest that these cell subgroups are essential to capsule development through pro-fibrotic activity, as Wnt11-knockout mice display significantly thinner capsules with a lower cellularity and loosely arranged fibers compared to the capsule of Bl6-mice after 3 months.

Publications: Lako M, Strachan T, Bullen P, Wilson D I, Robson S C, Lindsay S (December 1998). “Isolation, characterisation and embryonic expression of WNT11, a gene which maps to 11q13.5 and has possible roles in the development of skeleton, kidney and lung”. Gene. 219 (1-2): 101-10. doi: 10.1016/50378-1119(98)00393-X.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” to another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A biomedical device comprising an effective amount of a composition containing a Wnt11 inhibitor for preventing, inhibiting or treating capsular fibrosis.

2. A biomedical device comprising an effective amount of a composition containing a monoterpenoid for preventing, inhibiting or treating capsular fibrosis.

3. A biomedical device comprising an effective amount of a composition containing 1,8-cineol for preventing, inhibiting or treating capsular fibrosis.

4. The biomedical device of claim 1 wherein the biomedical device is an implant configured to prevent, inhibit, or treat capsular fibrosis when the biomedical device is implanted into a body of an individual having or at risk of having capsular fibrosis.

5. The biomedical device of claim 4 wherein the implant is configured to stay in the body at least 2 weeks, at least 4 weeks, at least 8 weeks, at least 3 months, at least 6 months, or at least one year.

6. The biomedical device of claim 1 wherein the biomedical device is an insert configured to prevent, inhibit, or treat capsular fibrosis when the biomedical device is inserted into a body of an individual having or at risk of having capsular fibrosis.

7. The biomedical device of claim 1 wherein the biomedical device is an interface configured to prevent, inhibit, or treat capsular fibrosis when placed on a body of an individual having or at risk of having capsular fibrosis.

8. The biomedical device of claim 1 wherein the device is configured to administer the composition systemically.

9. The biomedical device of claim 1 wherein the device is configured to administer the composition locally.

10. The biomedical device of claim 1 wherein the device is configured to administer the composition intraarterially, intraarticularly, intramuscularly, intraocularly, intraperitoneally, intravenously, or subcutaneously.

11. The biomedical device of claim 1 wherein the device is configured to administer the composition buccally, intranasally, orally, intrarectally, intravaginally, or sublingually.

12. The biomedical device of claim 1 wherein the device is configured to administer the composition iontophoretically, topically, or transdermally.

13. The biomedical device of claim 1 wherein the device is coated with the composition.

14. The biomedical device of claim 1 wherein the device is impregnated with the composition.

15. The biomedical device of claim 1 wherein the composition is covalently attached to the device.

16. The biomedical device of claim 1 wherein the composition is non-covalently attached to the device.

17. The biomedical device of claim 1 wherein the device comprises a silicone implant.

18. A method of preventing, inhibiting, or treating capsular fibrosis in an individual in need thereof comprising:
selecting an individual that has or is at risk of having fibrosis; and
administering to the individual a composition having an effective amount of a Wnt11 inhibitor and thereby preventing, inhibiting, or treating capsular fibrosis in the individual.

19. A method of preventing, inhibiting, or treating capsular fibrosis in an individual in need thereof comprising:
selecting an individual that has or is at risk of having capsular fibrosis; and
administering to the individual a composition having an effective amount of a monoterpenoid and thereby preventing, inhibiting, or treating capsular fibrosis in the individual.

20. A method of preventing, inhibiting, or treating capsular fibrosis in an individual in need thereof comprising:
selecting an individual that has or is at risk of having capsular fibrosis; and
administering to the individual a composition having an effective amount of 1,8-cineol and thereby preventing, inhibiting, or treating capsular fibrosis in the individual.

21. A method of preventing, inhibiting, or treating capsular fibrosis in an individual in need thereof comprising:
selecting an individual that has or is at risk of having capsular fibrosis; and
administering to the individual a composition having an effective amount of macrophage inhibiting activity and thereby preventing, inhibiting, or treating capsular fibrosis in the individual.

22. A method of preventing, inhibiting, or treating capsular fibrosis in an individual in need thereof comprising:
selecting an individual that has or is at risk of having capsular fibrosis; and
administering to the individual a composition having an effective amount of an M2 macrophage inhibiting activity and thereby preventing, inhibiting, or treating capsular fibrosis in the individual.

23. The method of claim 18 wherein the composition is administered intraarterially, intraarticularly, intramuscularly, intraocularly, intraperitoneally, intravenously, or subcutaneously.

24. The method of claim 18 wherein the composition is administered buccally, intranasally, orally, intrarectally, intravaginally, or sublingually.

25. The method of claim 18 wherein the composition is administered iontophoretically, topically, or transdermally.

26. The method of claim 18 wherein the composition is administered locally.

27. The method of claim 18 wherein the composition is administered systemically.

28. The method of claim 18 wherein the administering step comprises implanting an implant comprising the composition.

29. The method of claim 28 wherein the implant is further configured to treat a medical issue or cosmetic issue other than fibrosis.

30. The method of claim 18 wherein the administering step comprises inserting an insert comprising the composition.

31. The method of claim 18 wherein the administering step comprises placing a skin interface of a biomedical device on a skin of the individual.

32. The method of claim 18 wherein the composition prevents, inhibits, or reduces macrophage activity or differentiation by at least 10%, at least 20%, at least 30%, at least 40% or at least 50%.

33. The method of claim 18 wherein the composition prevents, inhibits, or reduces M2 macrophage activity or differentiation at least 10%, at least 20%, at least 30%, at least 40% or at least 50%.

34. The method of claim 18 wherein the composition prevents, inhibits, or reduces Cd36 macrophage activity or differentiation, Ccr2 macrophage activity or differentiation and/or Cd209 macrophage activity or differentiation at least 10%, at least 20%, at least 30%, at least 40% or at least 50%.

35. The method of claim 18, wherein the composition prevents, inhibits, or treats collagen deposition.

36. The method of claim 18, wherein the composition prevents, inhibits, or treats a foreign body response.

37. The method of claim 18, wherein the individual is human.
